SEQUENCE LISTING (3 sequences)

SEQ ID NO: 1 - DNA, 20 bp, Artificial Sequence
  Primer
atgccccaaccattgaagca

SEQ ID NO: 2 - DNA, 20 bp, Artificial Sequence
  Primer
ttccaatttcgggaagggca

SEQ ID NO: 3 - protein, 5 aa, Artificial Sequence
  Probe
GGGGS

(19) United States
(12) Patent Application Publication
(10) Pub. No.: US 2010/0297081 A1
(43) Pub. Date: Nov. 25, 2010

(54) PHARMACEUTICAL FORMULATION CONTAINING RECOMBINANT HUMAN SERUM ALBUMIN-INTERFERON ALPHA FUSION PROTEIN

(76) Inventors: Yanshan Huang, Zhejian Province (CN); Guochang Ma, Zhejian Province (CN); Tongying Wang, Zhejian Province (CN); Feihu Xu, Zhejian Province (CN)

Correspondence Address: JACOBSON HOLMAN PLLC, 400 SEVENTH STREET N.W., SUITE 600, WASHINGTON, DC, 20004, US

(21) Appl. No.: 12/809,298

(22) PCT Filed: Dec. 18, 2007
(86) PCT No.: PCT/CN07/03666

§ 371 Date: Jun. 18, 2010

(51) Int. Cl. A61K 38/21; A61P 31/14; A61P 31/20

(52) U.S. Cl. 424/85.7

(57) ABSTRACT

The present invention provides a pharmaceutical formulation containing a recombinant human serum albumin-interferon α fusion protein (rHSA-IFNα), said formulation is prepared by dissolving the fusion protein and a pharmaceutically acceptable stable excipient in a pharmaceutically acceptable buffer which pH ranges from 5.0 to 8.0. The recombinant human serum albumin-interferon α fusion protein concentration ranges from 0.1 mg/ml to 5 mg/ml. The stable excipient is glycine or methionine. The pharmaceutical formulation containing rHSA-IFNα has storage stability, which could act as immunomodulators for the treatment of viral infectious diseases, tumors and related diseases in the route of subcutaneous or intravenous administration.

### FIELD OF THE INVENTION

The present invention relates to a pharmaceutical formulation containing a recombinant human serum albumin-interferon alpha fusion protein (rHSA-IFNα), which can act as immunomodulators for the treatment of viral infectious diseases, tumors and related diseases in the route of subcutaneous or intravenous administration.

### BACKGROUND OF THE INVENTION

Interferon α (IFNα) is the most widely used antiviral drug in clinical treatment of hepatitis C, hepatitis B, cancer and AIDS-related Kaposi's sarcoma and other diseases. IFNα can inhibit the hepatitis B and hepatitis C virus replication and reduce plasma transaminase. However, as a small protein, IFNα is cleared from plasma quickly, its half-life is about 3 ˜8 hours after the injection, and, 24 hours later, the presence of IFNα in plasma cannot be detected. This is extremely unfavorable for the treatment.

IFNα is usually injected once a day or twice a week when it is used for the treatment of hepatitis. However, in most treatment period, the IFNα concentration in vivo is lower than the effective concentration. On the other hand, IFNα concentration is much higher than the effective concentration when its blood concentration reaches peak after administration, which would produce significant side effects. In order to increase IFNα half-life in vivo, a widely used method now is the PEG modification method. There are already about 40 kD PEG modified IFNα2a (Pegasys, Roche) and about 12 kD PEG modified IFNα2b (PEG-Intron, Schering-Plough) used in clinical treatment. These two products have significantly longer half-life than IFNα in vivo, and their PEG modification sites are in the lysine residues of interferon protein molecules. IFNαcontains 10 ˜11 lysine, thus it will form different isomers in the process of PEG modification. These isomers mixture will result in many different physiological responses. Although the method of using fixed-point mutation can introduce a cysteine (Q5C) and PEG modification occurs on the cysteine, which can be modified to achieve single fixed-point modification, the safety and efficacy of the IFNα mutant need further evaluation in human body.

Human serum albumin (HSA) is the main component in human serum, which plays a vital role to maintain osmotic pressure and plasma volume of the body. Human serum albumin is a non-glycosylated protein and its molecular weight is 6615 kD. Its renal clearance rate is very low in vivo and its half-life is 14 ˜20 days. It is also a natural carrier of body's factors and drug delivery. Studies show that the fusion protein expressed by the therapeutic protein gene linked with the human serum albumin gene can reduce the drug clearance rate in vivo, and extend the biological half-life. Yeh et al. found that the half-life of HSA-CD4 fusion protein in rabbits expressed in Kluyveromyces yeast extended 140 times longer than CD4 protein alone, and the half-life of HSA-IFNα fusion protein (albuferon) in monkeys expressed in Kluyveromyces yeast extended 18 times longer than IFNα alone. (Blaire L., et al., The Journal of Pharmacology And Experimental Therapeutics. 2002, 303: 540-548).

There are many literatures reporting preparation methods of rHSA-IFNα (Wu Jun, et al., China Patent No.: 01124110.1; Blaire L., et al., The Journal of Pharmacology And Experimental Therapeutics. 2002, 303: 540-548; Fu Yan, et al.: US Patent Application No. 20060051859). The corresponding fusion protein can be obtained by fusing human serum albumin gene to human interferon α gene and selecting appropriate recombinant expression method. In the structure of the fusion protein, the C-terminal of human serum albumin is fused directly or through a flexible linker peptide sequence to the N-terminal of human interferon α, or C-terminal of human interferon α is fused directly or through a flexible linker peptide sequence to the N-terminal of human serum albumin. The general formula of a flexible linker peptide sequence is [GlyGlyGlyGlySer]ₙ, n being an integer between 1 and 10, preferably n being an integer between 1 and 3, most preferably n being 1. In the fusion protein, said interferon α is selected from interferon α2a, interferon α1b, interferon α2b or interferon α con, preferably interferon α2b.

Recombinant human serum albumin-interferon α fusion protein overcomes the traditional interferon's shortcomings of multiple-dose injections in therapy, and has the following advantages of: 1) stimulating the body's immune response to viral infection; 2) extending the lifetime of interferon in vivo; and 3) enlarging and improve treatment effect, and reducing the potential side effects or toxicity of conventional interferon treatment.

However, as a kind of protein drug, the stability of rHSA-IFNα cannot compete with conventional chemical drugs (Panayotatos; Nikos, 1998, U.S. Pat. No. 5,846,935), because its activity in long-term storage will be affected by various environmental factors, such as high sensitivity to temperature, oxygen and UV. These factors may cause many physical or chemical changes, such as combination, aggregation and oxidation. The protein drug thus loses much of its activity. If the rHSA-IFNα stability in long-term storage cannot be guaranteed, it will lead to the changes of dose, then affect the treatment effect.

Therefore, developing a kind of pharmaceutical formulation containing rHSA-IFNαprotein which can be stably preserved and suitable for clinical use is extremely meaningful. However, there is yet no reported research about this.

### DETAILED DESCRIPTION OF THE INVENTION

The purpose of this invention is to provide a pharmaceutical formulation containing recombinant human serum albumin-interferon α fusion protein (rHSA-IFNα), which can be stably preserved and suit for practical clinical use.

This invention provides a pharmaceutical formulation containing recombinant human serum albumin-interferon α fusion protein, wherein said formulation comprises a recombinant human serum albumin-interferon α fusion protein (rHSA-IFNα) as an active ingredient, a pharmaceutically acceptable buffer which can be maintained a pH of 5.0 to 8.0 in aqueous solution and pharmaceutically acceptable excipients which can enhance the stability of rHSA-IFNα protein. The advantages of this invention is to enhance the rHSA-IFNα protein's physical and chemical stability and biological activities by adding a number of components that can be accepted by the human body, then providing a kind of pharmaceutical formulation suitable for clinical use, especially for injection. Such formulation can prevent the active ingredients (rHSA-IFNα protein) from invalidation lead by a number of factors such as container adsorption, degradation and oxidation, thus facilitating the formulation for the transportation, long-term preservation and clinical use.

Various literatures may be referred to regarding the preparation methods of various types of recombinant human serum albumin-interferon α fusion protein (Wu Jun, et al., China Patent No. 01124110.1; Blaire L., et al., The Journal of Pharmacology And Experimental Therapeutics. 2002, 303: 540-548; Fu Yan, et al.: US20060051859). The corresponding fusion protein can be obtained by fusing human serum albumin to a human interferon α gene and selecting appropriate recombinant expression methods. In the structure of the fusion protein, the C-terminal of human serum albumin is fused either directly or through a flexible linker peptide sequence to the N-terminal of human interferon α, or the C-terminal of human interferon α is fused either directly or through a flexible linker peptide sequence to the N-terminal of human serum albumin. The general formula of the flexible linker peptide sequence is [GlyGlyGlyGlySer]ₙ, n being an integer between 1 and 10, preferably n being an integer between 1 and 3, most preferably n being 1. In the fusion protein, said interferon α is selected from interferon α2a, interferon α 1b, interferon α2b or interferon α con, preferably interferon α2b.

In the formulation described above, the concentration of the recombinant human serum albumin-interferon-α fusion protein is 0.1-5 mg/ml, preferably 0.5˜2 mg/ml.

In the formulation described above, the stabilizing excipient can be added as needed, such as amino acids and sugars. In the present invention, the preferred stabilizing excipient is glycine or methionine, the mass concentration (excipient weight/volume of solution, w/v) is 1-4%; Preferably mass concentration of glycine is 1-4%, most preferably mass concentration of glycine is 2.3%.

In the formulation described above, the buffer suitable for this invention may be any buffer which can maintain a pH of between 5.0 and 8.0 in aqueous solution, selected from disodium hydrogen phosphate-citric acid buffer, phosphate buffer, tris(hydroxymethyl) amino methane hydrochloride (Tris-HCl) buffer, acetic acid-sodium acetate buffer, citric acid buffer, barbiturate buffer or succinate buffer; the concentration of buffer ranges from 5 mmol/L to 100 mmol/L, preferably 5 mmol/L to 30 mmol/L, most preferably 10 mmol/L. The pH of buffer ranges from 5.0 to 8.0, preferably 6.0 to 7.0, most preferably 6.5. Among them, preferred buffer is phosphate buffer, the concentration ranges from 5 mmol/L to 100 mmol/L, preferably 5 mmol/L to 30 mmol/L, most preferably 10 mmol/L. The pH of buffer ranges from 5.0 to 8.0, preferably 6.0 to 7.0, most preferably 6.5.

Preferably, the pharmaceutical formulation described above is prepared by dissolving a recombinant human serum albumin-interferon α2b fusion protein and glycine in a phosphate buffer whose pH ranges from 5.0 to 8.0 and the concentration ranges from 5 mmol/L to 100 mmol/L, said recombinant human serum albumin-interferon α2b fusion protein is prepared by linking human serum albumin directly or through a peptide linker which general formula is [GlyGlyGlyGlySer]ₙ with interferon, n being an integer between 1 and 10. The concentration of fusion protein ranges from 0.1 mg/ml to 5 mg/ml. Said glycine's concentration ranges from 1% to 4%.

More preferably, the pharmaceutical formulation described above is prepared by dissolving recombinant human serum albumin-interferon α2b fusion protein and glycine in phosphate buffer which a pH ranges from 6.0 to 7.0 and the concentration ranges from 5 mmol/L to 30 mmol/L. Said recombinant human serum albumin-interferon α2b fusion protein is prepared by linking human serum albumin directly or through a peptide linker which general formula is [GlyGlyGlyGlySer]ₙ with an interferon, n being an integer between 1 and 3. The concentration of fusion protein ranges from 0.5 mg/ml to 2 mg/ml, and said glycine's concentration ranges from 1% to 4%.

More preferably, the pharmaceutical formulation described above is prepared by dissolving recombinant human serum albumin-interferon α2b fusion protein and glycine in a phosphate buffer whose pH is 6.5 and the concentration is 10 mmol/L. Said recombinant human serum albumin-interferon α2b fusion protein is prepared by linking human serum albumin directly or through a linker peptide [GlyGlyGlyGlySer]ₙ with an interferon. The concentration of the fusion protein is 0.5 mg/ml, and said glycine's concentration is 2.3%.

If necessary, the pharmaceutical formulation described above can be prepared as a freeze-dried powder. Before being freeze-dried, the liquid pharmaceutical formulation is essentially an isotonic solution. The freeze-dried powder can be restored to an isotonic solution after adding an appropriate amount of water for injection.

The present invention also provides a method of using a pharmaceutical formulation containing a recombinant human serum albumin-interferon α2b fusion protein in the manufacture of a medicament for the treatment of viral hepatitis such as hepatitis C, hepatitis B, et al. The duck hepatitis B model experiments suggest that the recombinant human serum albumin-interferon α2b fusion protein injection has a good anti-HBV effect.

### BRIEF DESCRIPTION OF THE DRAWINGS

FIG. 1 shows SDS-PAGE electrophoresis of the samples in different pH buffer at 40° C. condition, which had been stored for two (2) weeks (a) and four (4) weeks (b);

FIG. 2 shows SDS-PAGE electrophoresis of the samples in different concentration at 40° C. condition, which had been stored for two (2) weeks (a) and four (4) weeks (b);

FIG. 3 shows SDS-PAGE electrophoresis of the samples in different concentration at 4° C. condition, which had been stored for one (1) year;

### EMBODIMENTS OF THE INVENTION

In order to improve the stability of pharmaceutical formulation containing rHSA-IFNα, an in-depth study was conducted and it was found that if adding at least one excipient selected from appropriate adjuvant (such as carbohydrates, amino acids and their derivatives, surfactants, et al.) and inorganic salts into the formulation, and selecting appropriate pH and rHSA-IFNα concentration, the object of this invention could be effectively achieved.

In the process of stability research of the rHSA-IFNα pharmaceutical formulation, the clarity test, SDS-PAGE electrophoresis detection, protein concentration, RP-HPLC and biological activity are selected as indicators to observe changes in rHSA-IFNα formulation. These methods could be obtained from the “Chinese Pharmacopoeia 2005 Edition”.

The present invention is further explained in the following examples. Said rHSA-IFNα2b in the examples as follows means the fusion protein prepared by the method published in the Chinese Patent No. 01124110.1 and there was a linker GlyGlyGlyGlySer between a human serum albumin and interferon α2b. Because the linker was very short, it is believed that there was little influence on the natural property of the fusion protein. Thus, to a person skilled in the art, the following experiment results would naturally applies to the fusion protein wherein human serum albumin linked directly with interferon.

### Example 1

### The Influence of pH on the Stability of rHSA-IFNα2b

The pH is an important factor in affecting the protein stability in the injectable formulations. The pH of formulation can be maintained by adding a suitable buffer salts such as phosphate, acetate, citrate, barbiturates, Tris (Tris(hydroxymethyl)aminomethane), borate, succinate, et al. In order to evaluate the stability of formulations under different pH conditions, the experiments were conducted in the following conditions:

Experiment conditions: 1 ml/bottle (protein concentration was 1 mg/ml), each group with different pH buffers.

(1) pH=4.0 (Acetic acid-sodium acetate buffer), 10 mmol/L

(2) pH=5.0 (Acetic acid-sodium acetate buffer), 10 mmol/L

(3) pH=6.5 (Phosphate buffer), 5 mmol/L

(4) pH=6.5 (Phosphate buffer), 10 mmol/L (5) pH=6.5 (Phosphate buffer), 100 mmol/L

(6) pH=7.5 (Phosphate buffer), 10 mmol/L

(7) pH=8.0 (Tris-HCl buffer), 10 mmol/L

The samples were placed in 40° C. incubators and in dark for four weeks, and analyzed every two weeks.

Detection methods: non-reducing SDS-PAGE electrophoresis.

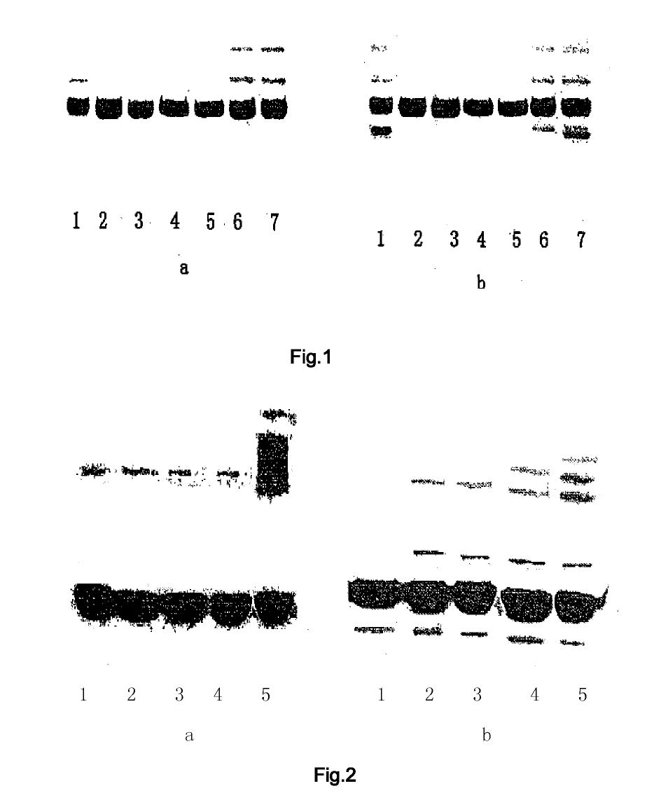

The results were shown in FIG. 1.

The 40° C. accelerated experiment showed that a low pH could inhibit the formation of protein aggregates, but it would speed up the protein degradation; a high pH would speed up the formation of aggregates, and it could not reduce the protein degradation. The samples were relatively stable at pH 5.0 and pH 6.5, and had less protein aggregates and degradation bands. Considering the pH of the human environment was about 7.0, for the purpose of being similar to the human environment, pH 6.5 was chosen as the condition of the sample formulation. Moreover, the results of phosphate buffer concentration ranged from 5 mmol/L to 100 mmol/L were essentially same. However, for the purpose of the osmotic pressure of the formulation being suitable for use in human body and maintaining a certain buffer capacity, the ultimate concentration of phosphate buffer in the formulations was 10 mmol/L.

### Example 2

### The Influence of Adding Different Excipients on the Stability of rHSA-IFNα2b

The general excipients suitable for protein formulations included albumin, sugars, amino acids, surfactants, metal chelating agents, et al. The present invention selected a number of suitable excipients to screen out which was the best one. Human serum albumin may contain potential blood-borne contaminants and the fusion protein already contained albumin, so they were not be considered.

The sugars suitable for the present invention may be selected from monosaccharides, oligosaccharides, polysaccharides, phospholipid and nucleotide derivatives, such as, glycerol, mannitol, sucrose, et al. These sugars could be added separately or used in combination.

The peptides, amino acids and derivatives suitable for the present invention may be selected from a group of substances as follows: glycine, alanine, serine, aspartic acid, glutamic acid, threonine, tryptophan, lysine, hydroxy lysine, histidine, arginine, cystine, cysteine, methionine, phenylalanine, leucine, isoleucine and their derivatives, et al.

In order to evaluate the influence of different excipients on the stability of the formulations, the excipients were screened according to the following conditions, and selected excipients were shown in Table 1 (in the table, the percentage of the concentration was mass concentration). Weighing the required amount of excipients or taking required amount of stock solution of the excipients, the excipients or the solution of excipients were added into the appropriate buffer (pH6.5 phosphate buffer), and then high concentrations of rHSA-IFNα2b dissolving in appropriate buffer (pH6.5 phosphate buffer) was added, using 1 mol/L HCl or 10% NaOH to adjust to the desired pH, then a certain volume of the sample solution comprising 10 mmol/L pH6.5 phosphate buffer and 0.5 mg/ml rHSA-IFNα2b was obtained. The sample solution was divided into two batches, each batch containing 12 samples and each sample containing 5 bottles (0.5 ml/bottle). One batch was used for the T=0 initial analysis, and then stored at 4° C. The other batch was stored at 40° C. for 4 months, and the samples were analyzed every month.

TABLE 1
Excipient Composition

| group | excipient | concentration |
|---|---|---|
| 1 | Glucose | 5% |
| 2 | Sucrose | 5% |
| 3 | Mannitol | 5% |
| 4 | Glycine −1 | 1% |
| 5 | Glycine −2 | 2.3% |
| 6 | Glycine −3 | 4% |
| 7 | Methionine | 2.3% |
| 8 | EDTA | 5 mmol/L |
| 9 | Tween-80 | 0.005% |
| 10 | Sucrose + Tween-80 | 5%, 0.005% |
| 11 | Mannitol + EDTA | 5%, 5 mmol/L |
| 12 | NaCl | 0.8% |

The stability results of different formulations were as follows:

(1) The Sample's Clarity Measurement

Table 2 and Table 3 showed there was no opacitas phenomenon occurred when the samples were stored at 4° C. and 40° C. for 4 months.

TABLE 2
Samples were stored at 4° C. for 4 months, and
the sample's clarity was measured every month

| Group | T-0 month | T-1 month | T-2 month | T-3 month | T-4 month |
|---|---|---|---|---|---|
| 1 | Colorless and | Colorless and | Colorless and | Colorless and | Colorless and |
| | clear | clear | clear | clear | clear |
| 2 | Colorless and | Colorless and | Colorless and | Colorless and | Colorless and |
| | clear | clear | clear | clear | clear |
| 3 | Colorless and | Colorless and | Colorless and | Colorless and | Colorless and |
| | clear | clear | clear | clear | clear |
| 4 | Colorless and | Colorless and | Colorless and | Colorless and | Colorless and |
| | clear | clear | clear | clear | clear |
| 5 | Colorless and | Colorless and | Colorless and | Colorless and | Colorless and |
| | clear | clear | clear | clear | clear |
| 6 | Colorless and | Colorless and | Colorless and | Colorless and | Colorless and |
| | clear | clear | clear | clear | clear |
| 7 | Colorless and | Colorless and | Colorless and | Colorless and | Colorless and |
| | clear | clear | clear | clear | clear |
| 8 | Colorless and | Colorless and | Colorless and | Colorless and | Colorless and |
| | clear | clear | clear | clear | clear |
| 9 | Colorless and | Colorless and | Colorless and | Colorless and | Colorless and |
| | clear | clear | clear | clear | clear |
| 10 | Colorless and | Colorless and | Colorless and | Colorless and | Colorless and |
| | clear | clear | clear | clear | clear |
| 11 | Colorless and | Colorless and | Colorless and | Colorless and | Colorless and |
| | clear | clear | clear | clear | clear |
| 12 | Colorless and | Colorless and | Colorless and | Colorless and | Colorless and |
| | clear | clear | clear | clear | clear |

TABLE 3
Samples were stored at 40° C. for 4 months, and
the sample's clarity was measured every month

| Group | T-0 month | T-1 month | T-2 month | T-3 month | T-4 month |
|---|---|---|---|---|---|
| 1 | Colorless and | Colorless and | Colorless and | Colorless and | Colorless and |
| | clear | clear | clear | clear | clear |
| 2 | Colorless and | Colorless and | Colorless and | Colorless and | Colorless and |
| | clear | clear | clear | clear | clear |
| 3 | Colorless and | Colorless and | Colorless and | Colorless and | Colorless and |
| | clear | clear | clear | clear | clear |
| 4 | Colorless and | Colorless and | Colorless and | Colorless and | Colorless and |
| | clear | clear | clear | clear | clear |
| 5 | Colorless and | Colorless and | Colorless and | Colorless and | Colorless and |
| | clear | clear | clear | clear | clear |
| 6 | Colorless and | Colorless and | Colorless and | Colorless and | Colorless and |
| | clear | clear | clear | clear | clear |
| 7 | Colorless and | Colorless and | Colorless and | Colorless and | Colorless and |
| | clear | clear | clear | clear | clear |
| 8 | Colorless and | Colorless and | Colorless and | Colorless and | Colorless and |
| | clear | clear | clear | clear | clear |
| 9 | Colorless and | Colorless and | Colorless and | Colorless and | Colorless and |
| | clear | clear | clear | clear | clear |
| 10 | Colorless and | Colorless and | Colorless and | Colorless and | Colorless and |
| | clear | clear | clear | clear | clear |
| 11 | Colorless and | Colorless and | Colorless and | Colorless and | Colorless and |
| | clear | clear | clear | clear | clear |
| 12 | Colorless and | Colorless and | Colorless and | Colorless and | Colorless and |
| | clear | clear | clear | clear | clear |

(2) Electrophoresis Measurement

The samples were measured by using SDS-PAGE method every month. Table 4 showed there was no degradation and aggregates occurred when the samples were stored at 4° C. for 4 months. Table 5 showed that only the samples containing glycine or methionine were relatively stable when the sample were stored at 40° C. under accelerated conditions. The degradation and aggregates occurred until 4 months later.

TABLE 4
Samples were stored at 4° C. for 4 months, and
the samples were measured by using SDS-PAGE every month

| Group | T-0 month | T-1 month | T-2 month | T-3 month | T-4 month |
|---|---|---|---|---|---|
| 1 | No | No | No | No | No |
| | degradation | degradation | degradation | degradation | degradation |
| | and no | and no | and no | and no | and no |
| | aggregates | aggregates | aggregates | aggregates | aggregates |
| 2 | No | No | No | No | No |
| | degradation | degradation | degradation | degradation | degradation |
| | and no | and no | and no | and no | and no |
| | aggregates | aggregates | aggregates | aggregates | aggregates |
| 3 | No | No | No | No | No |
| | degradation | degradation | degradation | degradation | degradation |
| | and no | and no | and no | and no | and no |
| | aggregates | aggregates | aggregates | aggregates | aggregates |
| 4 | No | No | No | No | No |
| | degradation | degradation | degradation | degradation | degradation |
| | and no | and no | and no | and no | and no |
| | aggregates | aggregates | aggregates | aggregates | aggregates |
| 5 | No | No | No | No | No |
| | degradation | degradation | degradation | degradation | degradation |
| | and no | and no | and no | and no | and no |
| | aggregates | aggregates | aggregates | aggregates | aggregates |
| 6 | No | No | No | No | No |
| | degradation | degradation | degradation | degradation | degradation |
| | and no | and no | and no | and no | and no |
| | aggregates | aggregates | aggregates | aggregates | aggregates |
| 7 | No | No | No | No | No |
| | degradation | degradation | degradation | degradation | degradation |
| | and no | and no | and no | and no | and no |
| | aggregates | aggregates | aggregates | aggregates | aggregates |
| 8 | No | No | No | No | No |
| | degradation | degradation | degradation | degradation | degradation |
| | and no | and no | and no | and no | and no |
| | aggregates | aggregates | aggregates | aggregates | aggregates |
| 9 | No | No | No | No | No |
| | degradation | degradation | degradation | degradation | degradation |
| | and no | and no | and no | and no | and no |
| | aggregates | aggregates | aggregates | aggregates | aggregates |
| 10 | No | No | No | No | No |
| | degradation | degradation | degradation | degradation | degradation |
| | and no | and no | and no | and no | and no |
| | aggregates | aggregates | aggregates | aggregates | aggregates |
| 11 | No | No | No | No | No |
| | degradation | degradation | degradation | degradation | degradation |
| | and no | and no | and no | and no | and no |
| | aggregates | aggregates | aggregates | aggregates | aggregates |
| 12 | No | No | No | No | No |
| | degradation | degradation | degradation | degradation | degradation |
| | and no | and no | and no | and no | and no |
| | aggregates | aggregates | aggregates | aggregates | aggregates |

TABLE 5
Samples were stored at 40° C. for 4 months
and detected by using SDS-PAGE every month

| group | T-0 month | T-1 month | T-2 month | T-3 month | T-4 month |
|---|---|---|---|---|---|
| 1 | No | No | degradation | degradation | degradation |
| | degradation | degradation | and | and | and |
| | and no | and no | aggregates | aggregates | aggregates |
| | aggregates | aggregates | | | |
| 2 | No | No | degradation | degradation | degradation |
| | degradation | degradation | and | and | and |
| | and no | and no | aggregates | aggregates | aggregates |
| | aggregates | aggregates | | | |
| 3 | No | No | No | degradation | degradation |
| | degradation | degradation | degradation | and | and |
| | and no | and no | and no | aggregates | aggregates |
| | aggregates | aggregates | aggregates | | |
| 4 | No | No | No | No | degradation |
| | degradation | degradation | degradation | degradation | and |
| | and no | and no | and no | and no | aggregates |
| | aggregates | aggregates | aggregates | aggregates | |
| 5 | No | No | No | No | degradation |
| | degradation | degradation | degradation | degradation | and |
| | and no | and no | and no | and no | aggregates |
| | aggregates | aggregates | aggregates | aggregates | |
| 6 | No | No | No | No | degradation |
| | degradation | degradation | degradation | degradation | and |
| | and no | and no | and no | and no | aggregates |
| | aggregates | aggregates | aggregates | aggregates | |
| 7 | No | No | No | degradation | degradation |
| | degradation | degradation | degradation | and | and |
| | and no | and no | and no | aggregates | aggregates |
| | aggregates | aggregates | aggregates | | |
| 8 | No | No | degradation | degradation | degradation |
| | degradation | degradation | and | and | and |
| | and no | and no | aggregates | aggregates | aggregates |
| | aggregates | aggregates | | | |
| 9 | No | degradation | degradation | degradation | degradation |
| | degradation | and | and | and | and |
| | and no | aggregates | aggregates | aggregates | aggregates |
| | aggregates | | | | |
| 10 | No | No | degradation | degradation | degradation |
| | degradation | degradation | and | and | and |
| | and no | and no | aggregates | aggregates | aggregates |
| | aggregates | aggregates | | | |
| 11 | No | No | degradation | degradation | degradation |
| | degradation | degradation | and | and | and |
| | and no | and no | aggregates | aggregates | aggregates |
| | aggregates | aggregates | | | |
| 12 | No | No | degradation | degradation | degradation |
| | degradation | degradation | and | and | and |
| | and no | and no | aggregates | aggregates | aggregates |
| | aggregates | aggregates | | | |

(3) RP-HPLC Measurement

The samples were measured by using RP-HPLC method every month. Table 5 showed samples stored at 4° C. for 4 months were stable, and the purity of samples did not change much. When the sample were stored at 40° C. under accelerated conditions, only the purity of the samples which contained glycine or methionine did not change much, the purity of other samples was significantly lowered.

TABLE 6
Samples were stored at 40° C. for 4 months,
and measured by using RP-HPLC every month

| | T-0 | T-1 | T-2 | T-3 | T-4 | T-1 | T-2 | T-3 | T-4 |
|---|---|---|---|---|---|---|---|---|---|
| | month | month | month | month | month | month | month | month | month |
| | 4° C. | 4° C. | 4° C. | 4° C. | 4° C. | 40° C. | 40° C. | 40° C. | 40° C. |
| group | (%) | (%) | (%) | (%) | (%) | (%) | (%) | (%) | (%) |
| 1 | 98.3 | 98.1 | 98.1 | 98.2 | 97.1 | 93.6 | 88.6 | 82.2 | 76.8 |
| 2 | 98.1 | 98.2 | 97.2 | 96.3 | 97.1 | 95.8 | 87.5 | 81.3 | 70.6 |
| 3 | 98.5 | 98.1 | 98.2 | 96.4 | 96.7 | 97.3 | 92.5 | 85.6 | 78.7 |
| 4 | 98.3 | 98.2 | 97.8 | 98.3 | 98.0 | 95.9 | 94.1 | 93.2 | 89.3 |
| 5 | 98.5 | 98.9 | 98.2 | 98.0 | 98.1 | 96.1 | 94.8 | 94.1 | 91.2 |
| 6 | 98.3 | 98.6 | 97.6 | 98.3 | 98.5 | 95.4 | 93.8 | 92.6 | 90.3 |
| 7 | 98.2 | 97.9 | 98.2 | 98.0 | 97.1 | 96.1 | 92.8 | 89.1 | 85.2 |
| 8 | 98.3 | 98.4 | 97.7 | 97.3 | 96.2 | 96.3 | 89.5 | 81.2 | 73.6 |
| 9 | 98.1 | 97.6 | 96.1 | 95.1 | 93.2 | 94.6 | 90.1 | 85.2 | 75.2 |
| 10 | 98.5 | 98.1 | 97.2 | 97.3 | 96.5 | 97.3 | 89.2 | 81.1 | 75.8 |
| 11 | 98.3 | 97.8 | 97.1 | 96.6 | 95.8 | 96.8 | 88.9 | 79.8 | 73.6 |
| 12 | 98.2 | 98.0 | 97.5 | 96.9 | 96.4 | 97.2 | 86.3 | 82.5 | 75.6 |

(4) Biological Activity (Potency)

The results described above showed that the samples containing glycine or methionine were the most stable. Therefore, only these samples were selected to evaluate the potency. Samples stored at 4° C. and 40° C. for 4 months were tested to measure their biological activity. The results (Table 7) showed that all the samples' potency were within the specified scope, suggesting that the chemical and physical degradation process did not significantly change the potency of protein activity, wherein the samples containing glycine were better than other samples, and different concentrations of glycine in samples had almost the same effect on protein's biological activity.

TABLE 7
biological activity detection of different samples were stored
at 4° C. and 40° C. respectively for 4 months

| group | Storage temperature | % potency |
|---|---|---|
| 4 | 4° C. | 102.2 |
| 4 | 40° C. | 85.3 |
| 5 | 4° C. | 105.2 |
| 5 | 40° C. | 89.5 |
| 6 | 4° C. | 103.4 |
| 6 | 40° C. | 86.7 |
| 7 | 4° C. | 101.5 |
| 7 | 40° C. | 82.3 |
| 12 | 4° C. | 102.2 |
| 12 | 40° C. | 58.6 |

The following conclusions could be drawn according to the results described above: sugars (including glucose, sucrose, mannitol) were adverse to the stability of rHSA-IFNα2b protein; surfactants could not reduce the aggregate formation; EDTA had no effect on the stability of rHSA-IFNα2b protein; but amino acids (glycine, methionine) had a good effect on the stability of formulation, wherein glycine was the best one. Therefore, glycine or methionine was chosen as the excipent of rHSA-IFNα2b formulation.

The above data also showed that glycine concentration ranged from 1% to 4% (w/w) in the formulation almost had the same effect on the protein stabilization. However, considering the osmotic pressure of the formulation should be similar to the physiological osmotic pressure, 2.3% (by weight) concentration of glycine was chosen as the optimal amount in the formulation.

### Example 3

### The Influence of Protein Concentration on the Stability of rHSA-IFNα2b

Protein concentration in an injectable pharmaceutical formulation was also an important factor affecting the stability of protein products. A low protein concentration would increase the formulation delivery volume, and the protein would be easily absorbed by the vessel wall. However, high protein concentration made it easier for protein to aggregate. In order to facilitate the capacity of the formulation suitable for practical use, and to maintain the stability of rHSA-IFNα2b protein in the formulation, a series of formulation stability tests in different protein concentrations were conducted according to the present invention.

Experimental conditions: 1 ml/bottle (10 mmol/L phosphate buffer, pH=6.5, 2.3% glycine), each group contained rHSA-IFNα2b of different concentrations.

(1) 0.1 mg/ml

(2) 0.5 mg/ml

(3) 1.0 mg/ml

(4) 2.0 mg/ml

(5) 5.0 mg/ml

The samples were placed in dark, 4° C. refrigerator and in dark, 40° C. constant temperature incubator separately to analyze the stability of the samples according to predetermined time.

Detection method: non-reducing SDS-PAGE electrophoresis (10 μg of each sample for electrophoresis)

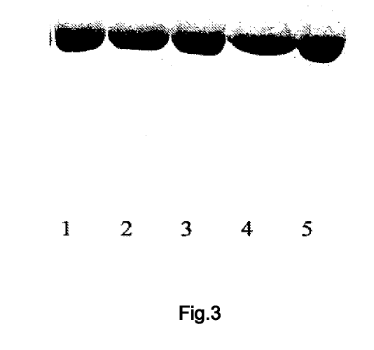

The results were shown in FIG. 2 and FIG. 3

The samples were stable when the sample's concentration ranged from 0.1 mg/ml to 5.0 mg/ml and placed in 4° C. refrigerator for 1 year. A 40° C. accelerated test showed that high protein concentration made it easier for protein to aggregate, which may affect their physical, chemical properties and biological activity. Considering the protein's stability and convenience to use, the most appropriate concentration of rHSA-IFNα2b was ranging from 0.5 mg/ml to 2 mg/ml.

According to Examples 1˜3, the following pharmaceutical formulation was ideal, which comprises: 0.1-5 mg/ml of rHSA-IFNα2b protein, preferably 0.5-2 mg/ml; an appropriate concentration of glycine or methionine, preferably 1-4% glycine, the most preferably glycine concentration was 2.3%; buffer was selected from phosphate buffer, Tris-HCl buffer, acetic acid-sodium acetate buffer, phosphate buffer was preferred, the concentration was 5-100 mmol/L, most preferable concentration was 10 mmol/L. Ultimately osmotic pressure of the formulations was 250-500 mOsm, pH was between 5-8, the most preferably was 6.5.

### Example 4

### The Preparation Process of the Formulation Injection Containing Recombinant Human Serum Albumin-Interferon α2b Fusion Protein

57.5 g of glycine was completely dissolved in 500 ml of human serum albumin-interferon α2b fusion protein stock solution which contained 10 mmol/L phosphate buffer (pH 6.5) and protein concentration was 2.5 mg/ml. Then 40 ml 0.5 mol/L phosphate buffer (pH 6.5) was added. The pH was adjusted to 6.5 with 10% NaOH. Finally, an appropriate amount of water for injection was added to make the final formulation volume to 2500 ml. The formulation was mixed and sterile filtered with 0.22 μm membrane, then packed in the ampoules. The final formulation comprises: 0.5 mg/ml recombinant human serum albumin-interferon α2b fusion protein, 10 mmol/L phosphate buffer, 2.3% (by weight) glycine, and the pH was 6.5.

### Example 5

### The Stability Evaluation of the Formulation Injection Containing Recombinant Human Serum Albumin-Interferon α2b Fusion Protein

According the process disclosed in Example 4, several preferred formulation was prepared as follows:

(1) the formulation injection comprising 0.5 mg/ml recombinant human serum albumin-interferon α2b fusion protein, 10 mM Na₂HPO₄—NaH₂PO₄ pH6.5 buffer, 2.3% glycine.

(2) the formulation injection comprising 2.0 mg/ml recombinant human serum albumin-interferon α2b fusion protein, 10 mM Na₂HPO₄—NaH₂PO₄ pH6.5 buffer, 2.3% glycine.

(3) the formulation injection comprising 0.5 mg/ml recombinant human serum albumin-interferon α2b fusion protein, 10 mM Na₂HPO₄—NaH₂PO₄ pH6.5 buffer, 2.3% methionine.

(4) the formulation injection comprising 2.0 mg/ml recombinant human serum albumin-interferon α2b fusion protein, 10 mM Na₂HPO₄—NaH₂PO₄ pH6.5 buffer, 2.3% methionine.

Said four preferred formulations were stored respectively at 4° C. and 40° C. for 4 months. RP-HPLC measurement of the samples were carried out every month, and biological activity detection of the samples were carried out after 4 months.

TABLE 8
RP-HPLC detection of the samples stored at 4° C. and 40° C.

| | T-0 | T-1 | T-2 | T-3 | T-4 | T-1 | T-2 | T-3 | T-4 |
|---|---|---|---|---|---|---|---|---|---|
| | month | month | month | month | month | month | month | month | month |
| | 4° C. | 4° C. | 4° C. | 4° C. | 4° C. | 40° C. | 40° C. | 40° C. | 40° C. |
| group | (%) | (%) | (%) | (%) | (%) | (%) | (%) | (%) | (%) |
| 1 | 98.6 | 98.4 | 98.5 | 98.2 | 97.9 | 96.9 | 95.1 | 94.2 | 92.3 |
| 2 | 98.3 | 98.1 | 98.0 | 98.2 | 97.1 | 96.3 | 94.4 | 93.1 | 91.2 |
| 3 | 98.5 | 98.3 | 98.3 | 98.4 | 97.2 | 96.4 | 94.8 | 92.5 | 90.5 |
| 4 | 98.3 | 98.3 | 98.1 | 98.2 | 98.0 | 96.1 | 94.2 | 91.1 | 89.2 |

TABLE 9
Biological activity measurement of the samples stored
at 4° C. and 40° C. after 4 months

| group | 4° C. | 40° C. |
|---|---|---|
| 1 | 106.1 | 89.6 |
| 2 | 103.2 | 87.2 |
| 3 | 103.1 | 88.3 |
| 4 | 105.5 | 83.4 |

These Experimental data described above suggested that the rHSA-IFNα2b protein formulations had good stability.

### Example 6

### The use of Recombinant Human Serum Albumin-Interferon α2b Fusion Protein Injection in the Treatment of Hepatitis B

### 6.1. Preparation of Duck Hepatitis B Model

(1) Screening Duck Hepatitis B Positive Serum

According to duck hepatitis B virus (DHBV) sequence, a pair of primers for amplification were designed. Upstream primer: 5 ‘atg ccc caa cca ttg aag ca 3 ’, downstream primer: 5 ttc caa ttt cgg gaa ggg ca 3′. Three (3) Shaoxing ducks were drawn blood in sterile conditions and the serum was separated. A 50 μl of lysis buffer was added into 5 μl of serum. The solution was heated under 100° C. for 10 minutes, then quickly put on ice after centrifugation, which acted as template for later use.

PCR reaction mixture was prepared as follows: 5 μl 10×PCR buffer, 3 μl 2.5 mM MgCl₂, 5 μl 2 mM dNTP, upstream and downstream primers each 20 pmol, 1.25u Taq DNA polymerase and water were added to a total volume of 45 μl. The mixture was mixed with the 5 μl template as prepared above, then a drop of paraffin oil was added, the mixture was put on the PCR machine. PCR procedure was that the reaction mixture was heat to 95° C. pre-denaturing for 2 minutes, then denaturing at 94° C. for 30 seconds, annealing at 56° C. for 30 seconds, extension at 72° C. for 45 seconds, said later three steps were a cycle, repeating 30 cycles, finally extension at 72 for 10 minutes.

Negative control sample contained all the necessary composition needed by RT-PCR but without the template. PCR results were verified by gel electrophoresis: 10 μl PCR reaction product was mixed with a 2 μl sampling buffer, then they were added to a 1.5% agarose gel comb hole soaked in 1×TBE buffer (90 mM tris-boric acid; 2 mM EDTA pH8.0). 40V electrophoresis was carried out for 3 hours, then the results were observed in the UV detector (wavelength 300 nm). The PCR amplification results of three (3) Shaoxing ducks were positive and they had three positive bands. The negative control sample did not show positive bands. The No. 1 Shaoxing duck serum which had the highest concentration band was selected as the positive serum.

(2) Preparation of duck hepatitis B model: the healthy Cherry Valley ducklings were selected after they emerged from their shells, each was intravenous injected 100 μl of positive duck serum through the leg vein. Other ten (10) only 1 day-old ducklings were selected as a normal control group. 1-day old Cherry Valley ducklings were infected by positive duck serum. Two (2) weeks later, each duckling was drawn 0.3 ml blood from its leg vein. The samples were measured by a PCR assay. The PCR methods and gel electrophoresis were described above. The positive Cherry Valley ducklings were selected as duck hepatitis B model.

### 6.2. Anti-HBV Therapy

(1) Positive Cherry Valley ducks were randomly divided into five groups (60 ducks/group), recombinant human serum albumin-interferon α2b fusion protein injection were prepared as in Example 4, the dosage was small dose group (3 μg/kg), medium dose (10 μg/kg), high-dose group (40 μg/kg); lamivudine (5 mg/day) as a positive control group, and the saline control group.

There were 42 ducks in each treatment group and the ducks were treated 2 weeks later after infection. Recombinant human serum albumin-interferon α2b fusion protein injection and saline injection were subcutaneously administrated once every other week. Lamivudine tablets were ground into powder and dissolved in cold water, then mixed with feed. The food was feed to Cherry Valley ducks twice daily, once in the morning and once in the evening. Each group was continuously administrated for 3 months, then observed 3 months after stopping the treatment.

(2) 10 Cherry Valley ducks taken from each group were drawn venous blood after infection but prior to treatment (2 weeks after infection), treatment of 1 month (6 weeks after infection), treatment of 3 months (14 weeks after infection), 1 month after stopping treatment (18 weeks after infection), 2 months after stopping treatment 1 (22 weeks after infection), 3 months after stopping treatment (26 weeks after infection). Then, these ducklings were sacrificed.

6.3 Detection of Duck Hepatitis B Virus Titer with Semi-Quantitative PCR

A 50 μl lysis buffer was added into a 5 μl Cherry Valley duck serum, heated under 100° C. for 10 minutes, and put on ice after a quick centrifugation, which was used as a template for later use. A PCR reaction mixture was prepared as follows: 5 μl 10×PCR buffer, 3 μl 2.5 mM MgCl₂, 5 μl 2 mM dNTP, upstream and downstream primers each 20 pmol, 1.25u Taq DNA polymerase, and water was added to the total volume of 45 μl. 5 μl of template was added into the PCR reaction mixture and mixed, a drop of paraffin oil was added, then the mixture on the PCR machine was added. PCR procedure was that the reaction mixture was heat to 95° C. pre-denaturing for 2 minutes, then denaturing at 94° C. for 30 seconds, annealing at 56° C. for 30 seconds, extension at 72° C. for 45 seconds, said three later steps were a cycle, repeating 30 cycles, finally extension at 72° C. for 10 minutes.

The No. 1 Shaoxing duck positive serum was selected as a positive control group. There were five positive controls in each reaction. Negative control sample contains all the necessary composition needed by RT-PCR but without the template. A 10 μl PCR reaction product was mixed with a 2 μl sampling buffer, then they were added to the 1.5% agarose gel comb holes soaked in 1×TBE buffer (90 mM tris-boric acid; 2 mM EDTA pH8.0). Electrophoresis was carried out under 40V for 3 hours, then semi-quantitative analysis was conducted by a gel imaging analysis system to obtain the optical density scanning values of each band for statistical analysis. Each image was balanced by the average value of five positive controls.

6.4.5 Serum Titer Changes of Duck Hepatitis B Virus after Treatment

Table 10 showed that at the end of 3 months treatment, the serum titer of duck hepatitis B virus in each treatment group had declined, and lamivudine treatment group was the most obvious. One month after stopping treatment, the virus titer in the group treated by the recombinant human serum albumin-interferon α2b fusion protein injection continued to decline. Three months after stopping treatment, there was no rebound and the fusion protein still had the virus inhibition effect. However, the DHBV DNA titer in lamivudine treatment group rebounded significantly after stopping treatment.

TABLE 10
Serum titer changes of duck hepatitis B virus after treated by human serum albumin-interferon α2b fusion protein injection

| | | | | 1 month after | 2 months after | 3 months after |
|---|---|---|---|---|---|---|
| | Before | 1 month after | In the end of | stopping | stopping | stopping |
| | treatment | treatment | treatment | treatment | treatment | treatment |
| High dose | 10542.6 ± 6953.5 | 13002.8 ± 7495.6* | 10301.5 ± 6920.1 | 3325.4 ± 989.5*Δ | 4345.1 ± 4133.4* | 4198.8 ± 4524.8 |
| Medium dose | 10891.8 ± 8953.1 | 13124 ± 9678.9 | 13013.4 ± 10064.2 | 3701.9 ± 4994.3* | 4843.3 ± 978.3* | 4380.9 ± 3619.1 |
| Small dose | 11023.5 ± 1186.4 | 176358.1 ± 8704.8* | 12654.3 ± 6278.0 | 4998.3 ± 3965.3* | 5873.2 ± 2954.5* | 4563.6 ± 1153.4* |
| lamivudine | 12541.3 ± 8932.9 | 13002.9 ± 7995.6* | 9103.5 ± 4986.9 | 7998.5 ± 5114.3 | 8679.4 ± 7896.0* | 6125.4 ± 2257.8 |
| saline | 11201.3 ± 8021.3 | 24051.6 ± 9425.2 | 14053.8 ± 6942.7 | 8943.5 ± 7968.9 | 23123.9 ± 14255.7 | 13053.5 ± 11421.5 |

Compared to model group:
*p < 0.05; compared to lamivudine group:
Δp < 0.05

The duck hepatitis B model experiments suggested that the recombinant human serum albumin-interferon α2b fusion protein injection had a good anti-HBV effect.

1. A pharmaceutical formulation containing recombinant human serum albumin-interferon α fusion protein, characterized in that said formulation comprises a recombinant human serum albumin-interferon α fusion protein (rHSA-IFNα) as an active ingredient, a pharmaceutically acceptable buffer which can maintain a pH of 5.0 to 8.0 in aqueous solution and pharmaceutically acceptable excipients which enhance the stability of rHSA-IFNα protein.

2. The pharmaceutical formulation of claim 1, characterized in that in the structure of the fusion protein, the C-terminal of human serum albumin is fused directly or through a flexible linker peptide sequence to the N-terminal of human interferon α, or C-terminal of human interferon α is fused directly or through a flexible linker peptide sequence to the N-terminal of human serum albumin.

3. The pharmaceutical formulation of claim 2, characterized in that the general formula of said flexible linker peptide sequence is [GlyGlyGlyGlySer]ₙ, n being an integer between 1 and 10.

4. The pharmaceutical formulation of claim 3, characterized in that the general formula of said flexible linker peptide sequence is [GlyGlyGlyGlySer]ₙ, n being an integer between 1 and 3.

5. The pharmaceutical formulation of claim 4, characterized in that the general formula of said flexible linker peptide sequence is [GlyGlyGlyGlySer]ₙ, n being 1.

6. The pharmaceutical formulation of claim 1, characterized in that said interferon α is selected from interferon α2a, interferon α1b, interferon α2b or interferon α con.

7. The pharmaceutical formulation of claim 6, characterized in that said interferon α is interferon α2b.

8. The pharmaceutical formulation of claim 1, characterized in that the concentration of said recombinant human serum albumin-interferon α fusion protein ranges from 0.1 mg/ml to 5 mg/ml.

9. The pharmaceutical formulation of claim 8, characterized in that the concentration of said recombinant human serum albumin-interferon α fusion protein ranges from 0.5 mg/ml to 2 mg/ml.

10. The pharmaceutical formulation of claim 1, characterized in that said pharmaceutically acceptable excipient which can enhance the stability of rHSA-IFNα protein is glycine or methionine, and the concentration ranges from 1% to 4% (w/w).

11. The pharmaceutical formulation of claim 10, characterized in that said pharmaceutically acceptable excipient which can enhance the stability of rHSA-IFNα protein is glycine, and the concentration ranges from 1% to 4% (w/w).

12. The pharmaceutical formulation of claim 11, characterized in that said pharmaceutically acceptable excipient which can enhance the stability of rHSA-IFNα protein is glycine, and the concentration is 2.3% (w/w).

13. The pharmaceutical formulation of claim 1, characterized in that said pharmaceutically acceptable buffer which can maintain a pH of 5.0 to 8.0 in aqueous solution is selected from disodium hydrogen phosphate-citric acid buffer, phosphate buffer, tris(hydroxymethyl) amino methane hydrochloride (Tris-HCl) buffer, acetic acid-sodium acetate buffer, citric acid buffer, barbiturate buffer or succinate buffer; the concentration ranges from 5 mmol/L to 100 mmol/L; and the pH of the buffer ranges from 5.0 to 8.0.

14. The pharmaceutical formulation of claim 13, characterized in that said pharmaceutically acceptable buffer which can maintain pH 5.0-8.0 in an aqueous solution is phosphate buffer, the concentration ranges from 5 mmol/L to 100 mmol/L, and the pH of the buffer ranges from 5.0 to 8.0.

15. The pharmaceutical formulation of claim 14, characterized in that said pharmaceutically acceptable buffer which can maintain pH 5.0-8.0 in an aqueous solution is phosphate buffer, the concentration ranges from 5 mmol/L to 30 mmol/L, and the pH of buffer ranges from 6.0 to 7.0.

16. The pharmaceutical formulation of claim 15, characterized in that said pharmaceutically acceptable buffer which can maintain a pH of 5.0 to 8.0 in an aqueous solution is phosphate buffer, the concentration is 10 mmol/L, and the pH is 6.5.

17. The pharmaceutical formulation of claim 1, characterized in that said pharmaceutical formulation is prepared by dissolving recombinant human serum albumin-interferon α2b fusion protein and glycine in phosphate buffer wherein pH ranges from 5.0 to 8.0 and the buffer concentration ranges from 5 mmol/L to 100 mmol/L, said recombinant human serum albumin-interferon α2b fusion protein is prepared by linking human serum albumin directly or through a peptide linker which general formula is [GlyGlyGlyGlySer]ₙ with interferon, n is an integer between 1 and 10, the concentration of fusion protein ranges from 0.1 mg/ml to 5 mg/ml, said glycine's concentration ranges from 1% to 4% (w/w).

18. The pharmaceutical formulation of claim 1, characterized in that said pharmaceutical formulation is prepared by dissolving recombinant human serum albumin-interferon α2b fusion protein and glycine in phosphate buffer wherein pH ranges from 6.0 to 7.0 and the buffer concentration ranges from 5 mmol/L to 30 mmol/L, said recombinant human serum albumin-interferon α2b fusion protein is prepared by linking human serum albumin directly or through a peptide linker which general formula is [GlyGlyGlyGlySer]ₙ with interferon, n is an integer between 1 and 3, the concentration of fusion protein ranges from 0.5 mg/ml to 2 mg/ml, said glycine's concentration ranges from 1% to 4% (w/w).

19. The pharmaceutical formulation of claim 1, characterized in that said pharmaceutical formulation is prepared by dissolving recombinant human serum albumin-interferon α2b fusion protein and glycine in phosphate buffer which pH is 6.5 and the buffer concentration is 10 mmol/l, said recombinant human serum albumin-interferon α2b fusion protein is prepared by linking human serum albumin directly or through a peptide linker [GlyGlyGlyGlySer] with interferon, the concentration of fusion protein is 0.5 mg/ml, said glycine's concentration is 2.3% (w/w).

20. A pharmaceutical formulation according to claim 1, characterized in that said pharmaceutical formulation can be prepared as a lyophilized powder.

21. A method of treatment of hepatitis C or hepatitis B of a subject comprising the step of administering to the subject a pharmaceutically effective amount of a pharmaceutical formulation containing recombinant human serum albumin-interferon fusion protein as claimed in claim 1.
